(19) United States
(12) Patent Application Publication
(10) Pub. No.: US 2024/0390700 A1
(43) Pub. Date: Nov. 28, 2024

(54) SYSTEMS AND METHODS FOR NOISE AND PATIENT DOSE OPTIMIZATION VIA DYNAMIC X-RAY MODULATION

(71) Applicant: Medtronic Navigation, Inc., Lafayette, CO (US)

(72) Inventors: Shuanghe Shi, Southborough, MA (US); Erica C. Freund, Cambridge, MA (US); Patrick A. Helm, Canton, MA (US); Michael Philip Marrama, Ayer, MA (US)

(21) Appl. No.: 18/670,685

(22) Filed: May 21, 2024

Related U.S. Application Data
(60) Provisional application No. 63/469,309, filed on May 26, 2023.

(51) Int. Cl. A61N 5/10; G16H 50/20

(52) U.S. Cl. CPC A61N 5/1031; G16H 50/20; A61N 2005/1058

(57) ABSTRACT

A system according to at least one embodiment of the present disclosure includes: an imaging source; an imaging detector; a processor; and a memory coupled to the processor and storing data thereon that, when executed by the processor, enable the processor to: initiate a multi-dimensional scan of patient anatomy that includes the imaging source emitting a radiation beam that is received by the imaging detector; receive radiation beam information associated with the radiation beam received by the imaging detector; compare the radiation beam information to a beam threshold value; and instruct, based on the comparing, the imaging source to adjust at least one beam parameter of the radiation beam such that the radiation beam information is compliant with the beam threshold value.

### CROSS-REFERENCE TO RELATED APPLICATION

This application claims the benefit of and priority to U.S. Provisional Patent Application No. 63/469,309 filed May 26, 2023, the entire disclosure of which is incorporated by reference herein.

### BACKGROUND

The present disclosure is generally directed to imaging, and relates more particularly to surgical imaging.

Imaging may be used by a medical provider for diagnostic and/or therapeutic purposes. Multiple images of patient anatomy may be captured. Patient anatomy can change over time, particularly following placement of a medical implant in the patient anatomy.

### BRIEF SUMMARY

Example aspects of the present disclosure include:

A system according to at least one embodiment of the present disclosure comprises: an imaging source; an imaging detector; a processor; and a memory coupled to the processor and storing data thereon that, when executed by the processor, enable the processor to: initiate a multi-dimensional scan of patient anatomy that includes the imaging source emitting a radiation beam that is received by the imaging detector; receive radiation beam information associated with the radiation beam received by the imaging detector; compare the radiation beam information to a beam threshold value; and instruct, based on the comparing, the imaging source to adjust at least one beam parameter of the radiation beam such that the radiation beam information is compliant with the beam threshold value.

Any of the features herein, wherein the data, when processed by the processor, further enable the processor to: capture a plurality of images of the patient anatomy using the imaging source and the imaging detector at fixed beam parameter settings; and determine, based on signals from the imaging detector and information about an amount of radiation output at the fixed beam parameter settings, a modulation range of the radiation beam.

Any of the features herein, wherein the plurality of images comprises an anterior-posterior view image and a lateral view image of the patient anatomy.

Any of the features herein, wherein the data, when processed by the processor, further enable the processor to: predict, during the multi-dimensional scan and at a first time, an orientation of the imaging source and the imaging detector at a second time later than the first time; and adjust, based on the predicted orientation, the at least one beam parameter.

Any of the features herein, wherein the imaging source comprises a thermionic emission tube, a cold emission tube, or a nuclear isotope tube.

Any of the features herein, wherein the at least one beam parameter comprises at least one of an amplitude and an amount of current.

Any of the features herein, wherein the multi-dimensional scan comprises at least one of the imaging source and the imaging detector revolving about a first axis that passes through the patient anatomy and the imaging source and the imaging detector moving along a direction of the first axis.

Any of the features herein, wherein the beam threshold value is based on a configuration of the imaging detector.

Any of the features herein, wherein the at least one beam parameter is adjusted by changing a beam filtration parameter of a collimator connected to the imaging source.

A system according to at least one embodiment of the present disclosure comprises: a processor; and a memory coupled to the processor and storing data thereon that, when processed by the processor, enable the processor to: initiate a multi-dimensional scan of patient anatomy that includes a source emitting a radiation beam that is received by a detector; receive radiation beam information associated with the radiation beam; compare the radiation beam information to a beam threshold; and instruct, based on the comparing, the source to adjust at least one beam parameter of the radiation beam such that the radiation beam information is compliant with the beam threshold.

Any of the features herein, wherein the data, when processed by the processor, further enable the processor to: capture a plurality of images of the patient anatomy using the source and the detector at fixed beam parameter settings; and adjust, based on signals from the detector and information about an amount of radiation output at the fixed beam parameter settings, a modulation range of the radiation beam.

Any of the features herein, wherein the plurality of images comprises an anterior-posterior view image and a lateral view image of the patient anatomy.

Any of the features herein, wherein the data, when processed by the processor, further enable the processor to: predict, during the multi-dimensional scan and at a first time, an orientation of the source and the detector at a second time later than the first time; and adjust, based on the predicted orientation, the at least one beam parameter.

Any of the features herein, wherein the source comprises a thermionic emission tube, a cold emission tube, or a nuclear isotope tube.

Any of the features herein, wherein the at least one beam parameter comprises at least one of an amplitude and an amount of current.

Any of the features herein, wherein the multi-dimensional scan comprises at least one of the source and the detector revolving about a first axis that passes through the patient anatomy and the source and the detector moving along a direction of the first axis.

Any of the features herein, wherein the beam threshold is based on a configuration of the detector.

Any of the features herein, wherein the at least one beam parameter is adjusted by changing a beam filtration parameter on a collimator connected to the source.

An apparatus according to at least one embodiment of the present disclosure comprises: a source that emits a radiation beam; a detector; a processor coupled to at least one of the source and the detector; and a memory coupled to the processor and storing data thereon that, when executed by the processor, enable the processor to: initiate a multi-dimensional scan of patient anatomy that includes the detector receiving the radiation beam after the radiation beam has passed through the patient anatomy; receive radiation beam information associated with the radiation beam received at the detector; compare the radiation beam information to a beam threshold value; and instruct, based on the comparing, the source to adjust at least one parameter of the radiation beam such that the radiation beam information is compliant with the beam threshold value.

Any of the features herein, wherein the data, when processed by the processor, further enable the processor to: capture a plurality of images of the patient anatomy using the source and the detector at fixed beam parameter settings; and determine, based on signals from the detector and information about an amount of radiation output at the fixed beam parameter settings, a modulation range of the radiation beam.

Any of the features herein, wherein the plurality of images comprises an anterior-posterior view image and a lateral view image of the patient anatomy.

Any of the features herein, wherein the data, when processed by the processor, further enable the processor to: predict, during the multi-dimensional scan and at a first time, an orientation of the source and the detector at a second time later than the first time; and adjust, based on the predicted orientation, the at least one parameter.

Any of the features herein, wherein the at least one parameter is adjusted by changing a beam filtration parameter of a collimator connected to the source.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present disclosure will become apparent to those skilled in the art upon consideration of the embodiment descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1A is a block diagram of aspects of a system according to at least one embodiment of the present disclosure;

FIG. 1B is a schematic of aspects of an imaging apparatus according to at least one embodiment of the present disclosure;

FIG. 1C is a schematic of the aspects of the imaging apparatus with imaging components in a first orientation according to at least one embodiment of the present disclosure;

FIG. 1D is a schematic of the aspects of the imaging apparatus with the imaging components in a second orientation according to at least one embodiment of the present disclosure;

FIG. 1E is a schematic of aspects of the imaging apparatus with the imaging components in an asymmetric configuration according to at least one embodiment of the present disclosure;

FIG. 1F is an alternative schematic view of the aspects of the imaging apparatus according to at least one embodiment of the present disclosure;

FIG. 2 is a flowchart according to at least one embodiment of the present disclosure;

FIG. 3 is a flowchart according to at least one embodiment of the present disclosure;

FIG. 4 is a flowchart according to at least one embodiment of the present disclosure; and

FIG. 5 is a flowchart according to at least one embodiment of the present disclosure.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia Geforce RTX 2000-series processors, Nvidia Geforce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

Cone Beam Computed Tomography (CBCT) systems such as an O-arm produce three-dimensional (3D) images of patient anatomy for treatment planning, surgical navigation, use in robotics, intraoperative reconciliation, and/or the like. The steps for performing CBCT imaging comprise positioning the patient at the isocenter of the imaging apparatus and acquiring x-ray projections of the volume of interest. The x-ray source and detector rotate 360 degrees along a circular path with the patient anatomy at the isocenter. The steps also include performing a reconstruction (e.g., using image processing) of the projection data. The projections include attenuation information of the anatomy in the x-ray beam. This information provides the volumetric representation of internal structures (e.g., patient anatomy). The reconstructed volume may then be displayed on a digital monitor for clinical use.

Sufficient transmission of the x-ray beam through the patient may be needed to overcome electronic and quantum noise in the flat panel x-ray detectors to produce a usable image. If there is insufficient signal at the detector, the image can have a poor signal to noise and contrast detection such that the user will not be able to distinguish anatomical features necessary for clinical purposes. Conversely, if the dynamic range of the detector is exceeded in certain projections, contrast is degraded due to pixel saturation. As a result, the x-ray technique parameters (e.g., kilovoltage peak (kVp), milliampere-seconds (mAs), pulse width, etc.) should be selected based on the acquisition geometry and/or patient or in-beam attenuation.

However, technique parameters are fixed over the 360 degree acquisition, while patient attenuation changes as a function of angle. For example, when imaging the thoracic region, the anterior-posterior (AP) view is less attenuating than the lateral (LAT) view. Thus, some projections will be over-exposed while some will be under-exposed. To optimize image quality, the technique parameters should be modulated over the 360 degree acquisition based on the per-projection attenuation of the beam.

According to at least one embodiment of the present disclosure, the x-ray beam may be modulated over a 3D acquisition. Minimum and maximum boundaries for signal level may be set. The boundaries may be based on the detector panel configuration for the corresponding imaging mode. AP and LAT images of the patient may be acquired at fixed technique parameter settings (e.g., fixed kV, fixed mA, etc.). The readouts of the detector signal may be used to determine the relative difference in attenuation and modulation range for 3D acquisition.

According to at least one embodiment of the present disclosure, the 3D image acquisition may be started. The signal level of each projection may be compared to the maximum and minimum boundaries. Based on the comparison, the beam parameters (e.g., mA, pulse width, etc.) may be adjusted for subsequent projections to maintain the signal within the bounds of maximum and minimum boundaries. The signal level may be repeatedly compared and the beam parameters may be repeatedly adjusted until the acquisition is complete.

The modulation sequence may require feedback to the x-ray generator and source assembly. In some cases, modulation may not be achievable for every projection due to, for example, hardware limitations to maintain fast acquisition time and x-ray beam uniformity. However, the second 180 degrees of projection data is redundant with the first 180 degrees. As a result, the modulation can be further optimized in the second 180 degrees of rotation based on the first 180 degrees of rotation. Furthermore, the threshold (e.g., minimum and maximum boundaries) can be configured based on the clinical objective. For instance, the threshold can be set low with a high detector gain mode to minimize patient dose.

Embodiments of the present disclosure provide technical solutions to one or more of the problems of (1) unnecessary overexposure to imaging radiation and (2) poor image quality due to varying attenuation.

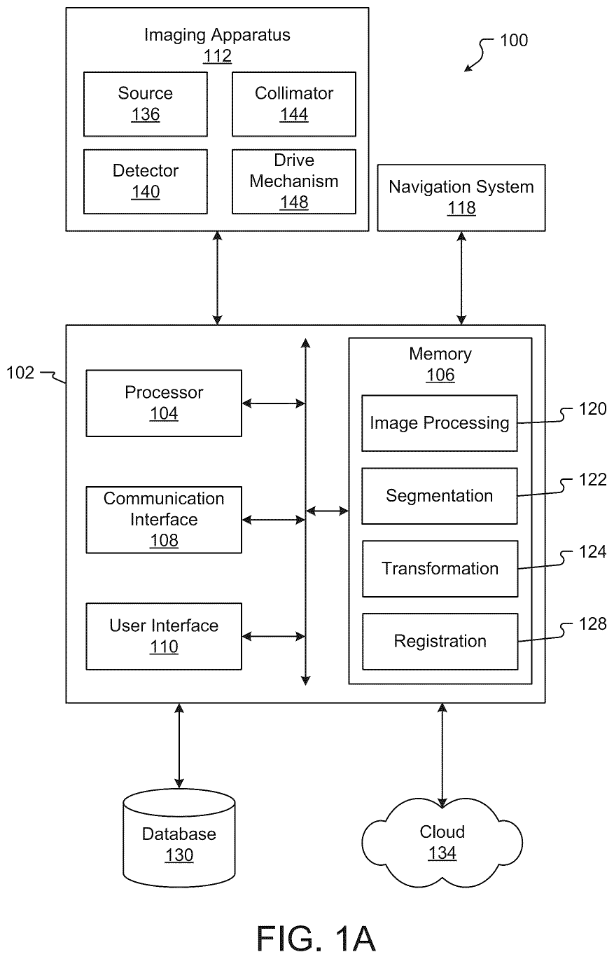

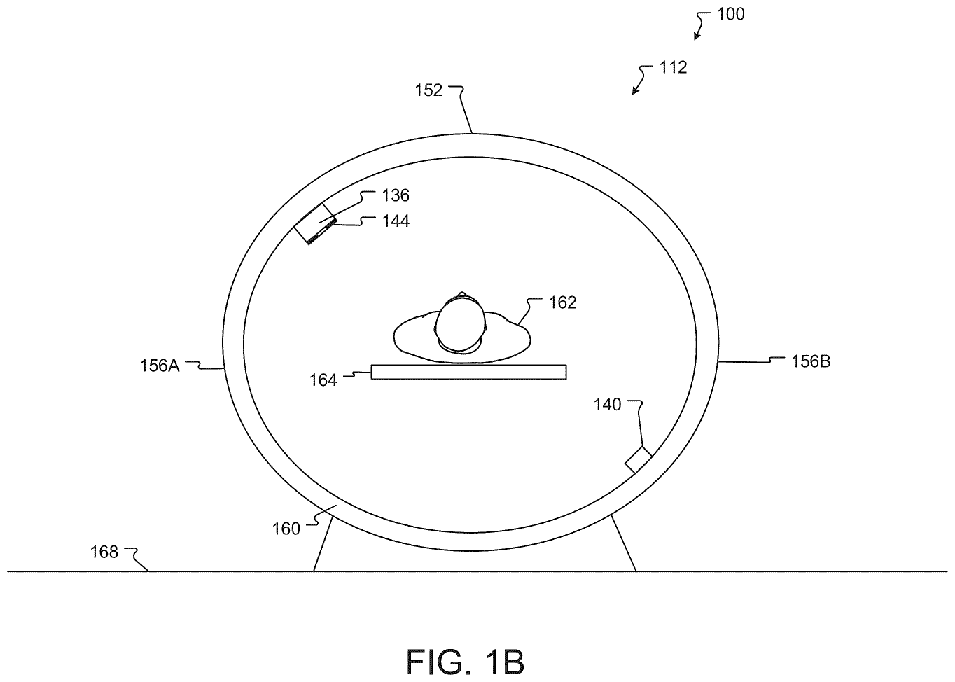

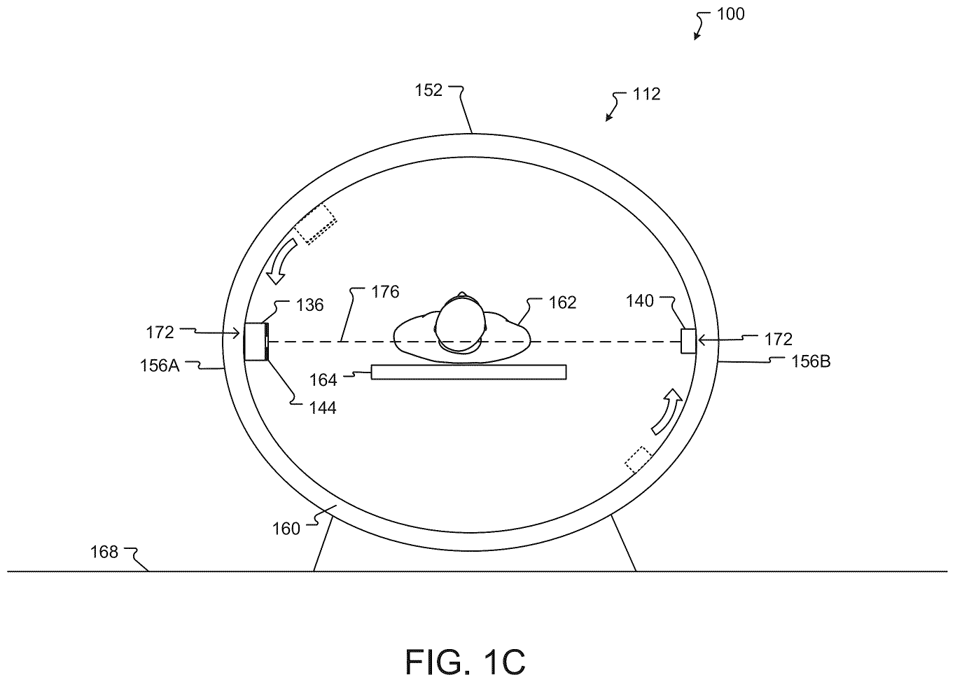

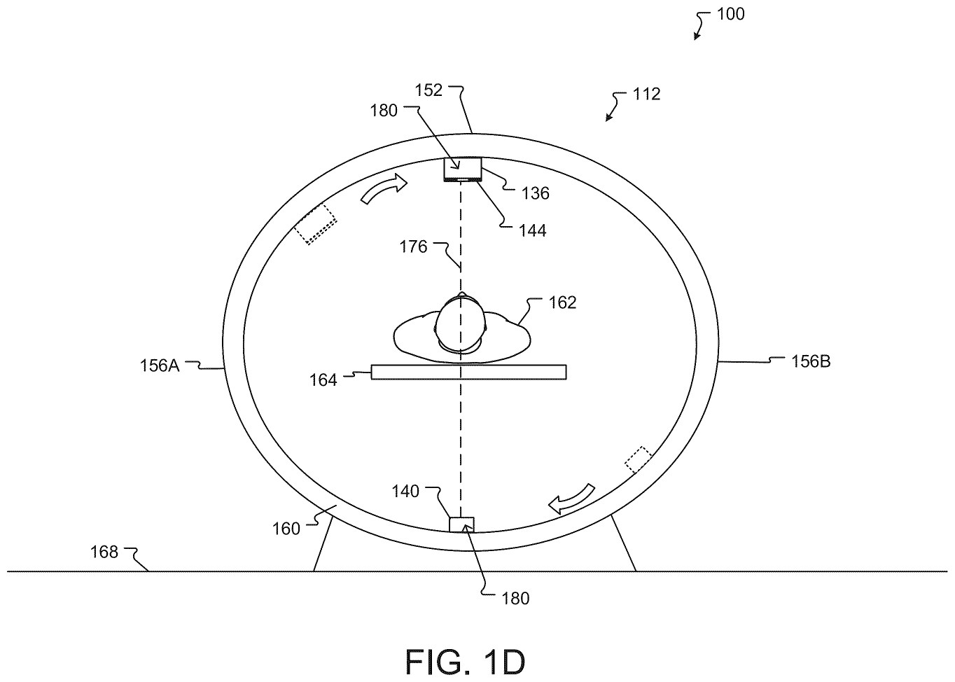

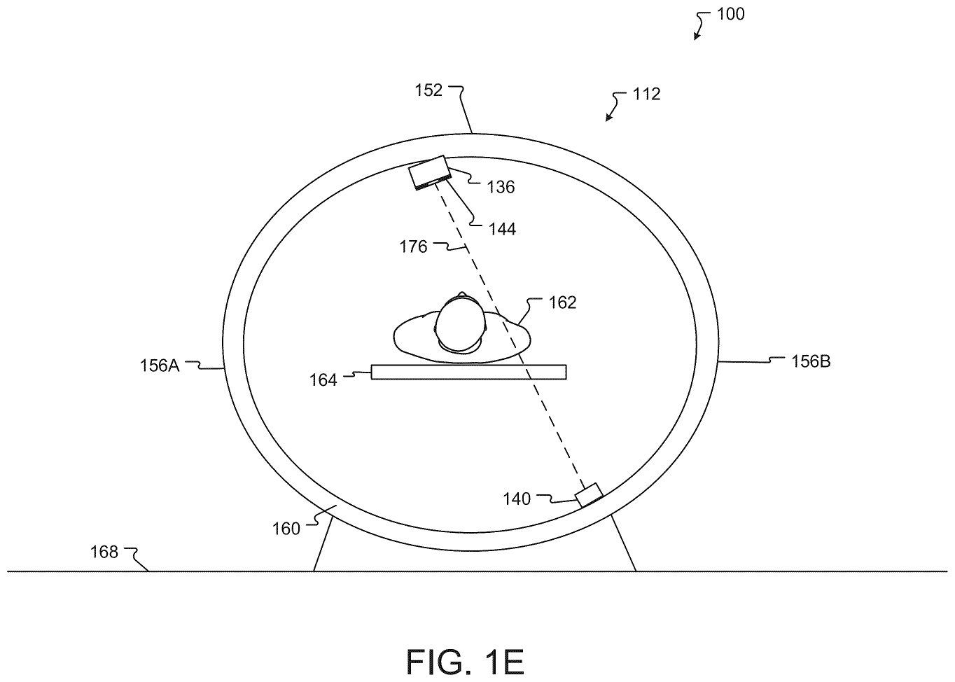

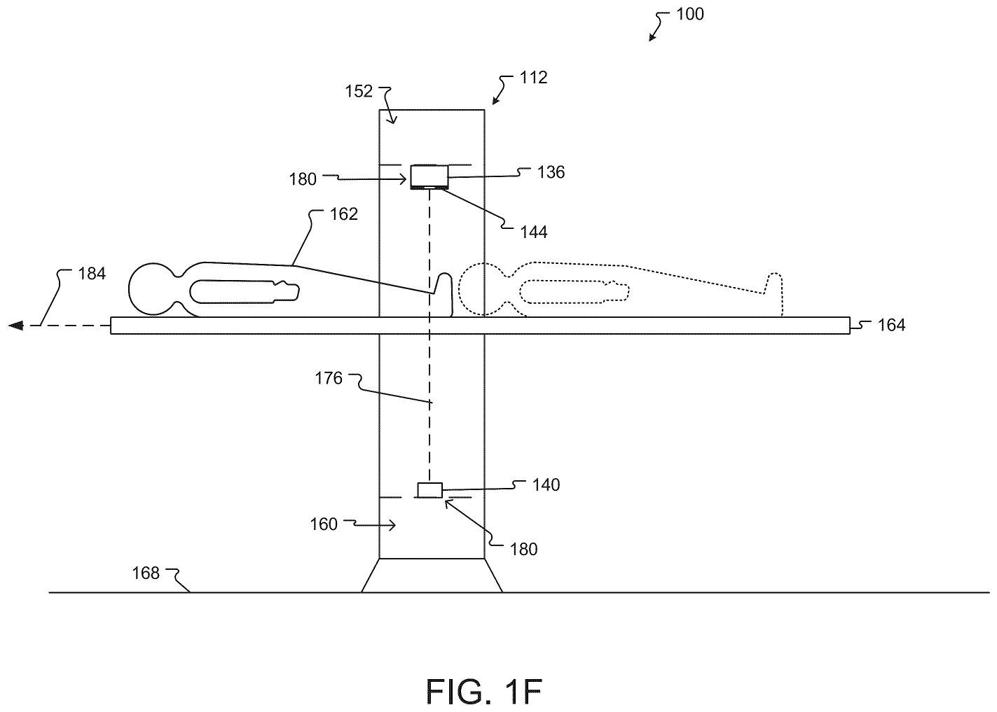

Turning first to FIG. 1A, a block diagram of a system 100 according to at least one embodiment of the present disclosure is shown. The system 100 may be used to modulate an x-ray beam; capture a multi-dimensional scan (e.g., a two-dimensional (2D) scan or a three-dimensional (3D) scan) of a patient; control, pose, and/or otherwise manipulate a surgical mount system, an imaging apparatus, and/or surgical tools attached thereto; and/or carry out one or more other aspects of one or more of the methods disclosed herein. The system 100 comprises a computing device 102, an imaging apparatus 112 comprising one or more imaging components, a navigation system 118, a database 130, and/or a cloud or other network 134. Systems according to other embodiments of the present disclosure may comprise more or fewer components than the system 100. For example, the system 100 may not include one or more components of the computing device 102, the database 130, and/or the cloud 134.

The computing device 102 comprises a processor 104, a memory 106, a communication interface 108, and a user interface 110. Computing devices according to other embodiments of the present disclosure may comprise more or fewer components than the computing device 102.

The processor 104 of the computing device 102 may be any processor described herein or any similar processor. The processor 104 may be configured to execute instructions stored in the memory 106, which instructions may cause the processor 104 to carry out one or more computing steps utilizing or based on data received from the imaging apparatus 112, the navigation system 118, the database 130, and/or the cloud 134.

The memory 106 may be or comprise RAM, DRAM, SDRAM, other solid-state memory, any memory described herein, or any other tangible, non-transitory memory for storing computer-readable data and/or instructions. The memory 106 may store information or data useful for completing, for example, any step of the methods 200, 300, 400, and/or 500 described herein, or of any other methods. The memory 106 may store, for example, instructions and/or machine learning models that support one or more functions of the imaging apparatus 112 and/or the navigation system 118. For instance, the memory 106 may store content (e.g., instructions and/or machine learning models) that, when executed by the processor 104, enable image processing 120, segmentation 122, transformation 124, and/or registration 128. Such content, if provided as in instruction, may, in some embodiments, be organized into one or more applications, modules, packages, layers, or engines. Alternatively or additionally, the memory 106 may store other types of content or data (e.g., machine learning models, artificial neural networks, deep neural networks, etc.) that can be processed by the processor 104 to carry out the various method and features described herein. Thus, although various contents of memory 106 may be described as instructions, it should be appreciated that functionality described herein can be achieved through use of instructions, algorithms, and/or machine learning models. The data, algorithms, and/or instructions may cause the processor 104 to manipulate data stored in the memory 106 and/or received from or via the imaging apparatus 112, the database 130, and/or the cloud 134.

The computing device 102 may also comprise a communication interface 108. The communication interface 108 may be used for receiving image data or other information from an external source (such as the imaging apparatus 112, the navigation system 118, the database 130, the cloud 134, and/or any other system or component not part of the system 100), and/or for transmitting instructions, images, or other information to an external system or device (e.g., another computing device 102, the imaging apparatus 112, the navigation system 118, the database 130, the cloud 134, and/or any other system or component not part of the system 100). The communication interface 108 may comprise one or more wired interfaces (e.g., a USB port, an Ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some embodiments, the communication interface 108 may be useful for enabling the computing device 102 to communicate with one or more other processors 104 or computing devices 102, whether to reduce the time needed to accomplish a computing-intensive task or for any other reason.

The computing device 102 may also comprise one or more user interfaces 110. The user interface 110 may be or comprise a keyboard, mouse, trackball, monitor, television, screen, touchscreen, and/or any other device for receiving information from a user and/or for providing information to a user. The user interface 110 may be used, for example, to receive a user selection or other user input regarding any step of any method described herein. Notwithstanding the foregoing, any required input for any step of any method described herein may be generated automatically by the system 100 (e.g., by the processor 104 or another component of the system 100) or received by the system 100 from a source external to the system 100. In some embodiments, the user interface 110 may be useful to allow a surgeon or other user to modify instructions to be executed by the processor 104 according to one or more embodiments of the present disclosure, and/or to modify or adjust a setting of other information displayed on the user interface 110 or corresponding thereto.

Although the user interface 110 is shown as part of the computing device 102, in some embodiments, the computing device 102 may utilize a user interface 110 that is housed separately from one or more remaining components of the computing device 102. In some embodiments, the user interface 110 may be located proximate one or more other components of the computing device 102, while in other embodiments, the user interface 110 may be located remotely from one or more other components of the computer device 102.

The imaging apparatus 112 may be or comprise imaging components operable to image anatomical feature(s) (e.g., a bone, veins, tissue, etc.) and/or other aspects of patient anatomy to yield image data (e.g., image data depicting or corresponding to a bone, veins, tissue, etc.). “Image data” as used herein refers to the data generated or captured by an imaging apparatus 112, including in a machine-readable form, a graphical/visual form, and in any other form. In various examples, the image data may comprise data corresponding to an anatomical feature of a patient, or to a portion thereof. The image data may be or comprise a preoperative image, an intraoperative image, a postoperative image, or an image taken independently of any surgical procedure. In some embodiments, the imaging apparatus 112 may comprise a first imaging device may be used to obtain first image data (e.g., a first image) at a first time, and a second imaging device may be used to obtain second image data (e.g., a second image) at a second time after the first time. The imaging apparatus 112 may be capable of taking a 2D image or a 3D image to yield the image data. The imaging apparatus 112 may be or comprise, for example, an ultrasound scanner (which may comprise, for example, a physically separate transducer and receiver, or a single ultrasound transceiver), an O-arm, a C-arm, a G-arm, or any other device utilizing X-ray-based imaging (e.g., a fluoroscope, a CT scanner, or other X-ray machine), a magnetic resonance imaging (MRI) scanner, an optical coherence tomography (OCT) scanner, an endoscope, a microscope, an optical camera, a thermographic camera (e.g., an infrared camera), a radar system (which may comprise, for example, a transmitter, a receiver, a processor, and one or more antennae), or any other imaging apparatus 112 suitable for obtaining images of an anatomical feature of a patient. The imaging apparatus 112 may be contained entirely within a single housing, or may comprise a transmitter/emitter and a receiver/detector that are in separate housings or are otherwise physically separated.

In some embodiments, the imaging apparatus 112 may comprise more than one imaging device. For example, a first imaging device may provide first image data and/or a first image, and a second imaging device may provide second image data and/or a second image. In still other embodiments, the same imaging device may be used to provide both the first image data and the second image data, and/or any other image data described herein. The imaging apparatus 112 may be operable to generate a stream of image data. For example, the imaging apparatus 112 may be configured to operate with an open shutter, or with a shutter that continuously alternates between open and shut so as to capture successive images. For purposes of the present disclosure, unless specified otherwise, image data may be considered to be continuous and/or provided as an image data stream if the image data represents two or more frames per second.

In some embodiments, reference markers (e.g., navigation markers) may be placed on the imaging apparatus 112, or any other object in the surgical space. The reference markers may be tracked by the navigation system 118, and the results of the tracking may be used by the navigation system 118 and/or by an operator of the system 100 or any component thereof. In some embodiments, the navigation system 118 can be used to track other components of the system (e.g., imaging apparatus 112) and the processor 104 can be used by the navigation system 118 to adjust imaging components of the imaging apparatus 112.

The navigation system 118 may provide navigation during an operation. The navigation system 118 may be any now-known or future-developed navigation system, including, for example, the Medtronic StealthStation™ S8 surgical navigation system or any successor thereof. The navigation system 118 may include one or more cameras or other sensor(s) for tracking one or more reference markers, navigated trackers, or other objects within the operating room or other room in which some or all of the system 100 is located. The one or more cameras may be optical cameras, infrared cameras, or other cameras. In some embodiments, the navigation system 118 may comprise one or more electromagnetic sensors. In various embodiments, the navigation system 118 may be used to track a position and orientation (e.g., a pose) of the imaging apparatus 112 or components thereof and/or one or more surgical tools (or, more particularly, to track a pose of a navigated tracker attached, directly or indirectly, in fixed relation to the one or more of the foregoing). The navigation system 118 may include a display for displaying one or more images from an external source (e.g., the computing device 102, imaging apparatus 112, or other source) or for displaying an image and/or video stream from the one or more cameras or other sensors of the navigation system 118. The navigation system 118 may be configured to provide guidance to a surgeon or other user of the system 100 or a component thereof, or to any other element of the system 100 regarding, for example, a pose of one or more anatomical elements, whether or not a tool is in the proper trajectory, and/or how to move a tool into the proper trajectory to carry out a surgical task according to a preoperative or other surgical plan.

The database 130 may store information that correlates one coordinate system to another (e.g., one or more coordinate systems associated with the imaging apparatus 112 to a patient coordinate system and/or to a navigation coordinate system). The database 130 may additionally or alternatively store, for example, one or more surgical plans (including, for example, pose information about a target and/or image information about a patient's anatomy at and/or proximate the surgical site, for use the navigation system 118 and/or a user of the computing device 102 or of the system 100); one or more images useful in connection with a surgery to be completed by or with the assistance of one or more other components of the system 100; and/or any other useful information. The database 130 may be configured to provide any such information to the computing device 102 or to any other device of the system 100 or external to the system 100, whether directly or via the cloud 134. In some embodiments, the database 130 may be or comprise part of a hospital image storage system, such as a picture archiving and communication system (PACS), a health information system (HIS), and/or another system for collecting, storing, managing, and/or transmitting electronic medical records including image data.

The cloud 134 may be or represent the Internet or any other wide area network. The computing device 102 may be connected to the cloud 134 via the communication interface 108, using a wired connection, a wireless connection, or both. In some embodiments, the computing device 102 may communicate with the database 130 and/or an external device (e.g., a computing device) via the cloud 134.

With reference to FIGS. 1B-1D, aspects of the imaging apparatus 112 according to at least one embodiment of the present disclosure are shown. The imaging apparatus 112 may include one or more imaging components that enables generation of one or more images of patient anatomy. In one embodiment, the imaging apparatus 112 comprises an O-arm.

The imaging apparatus 112 comprises an upper wall or member 152, a lower wall or member 160, and a pair of sidewalls or members 156A, 156B. In some embodiments, the imaging apparatus 112 is fixed securable to an operating room wall 168 (such as, for example, a ground surface of an operating room or other room). In other embodiments, the imaging apparatus 112 may be releasably securable to the operating room wall 168 or may be a standalone component that is simply supported by the operating room wall 168.

A table 164 configured to support a patient 162 may be positioned orthogonally to the imaging apparatus 112, such that the table 164 extends in a first direction from the imaging apparatus 112. In some embodiments, the table 164 may be mounted to the imaging apparatus 112. In other embodiments, the table 164 may be releasably mounted to the imaging apparatus 112. In still other embodiments, the table 164 may not be attached to the imaging apparatus 112. In such embodiments, the table 164 may be supported and/or mounted to an operating room wall, for example. In embodiments where the table 164 is mounted to the imaging apparatus 112 (whether detachably mounted or permanently mounted), the table 164 may be mounted to the imaging apparatus 112 such that a pose of the table 164 relative to the imaging apparatus 112 is selectively adjustable.

The table 164 may be any operating table configured to support the patient 162 during a surgical procedure. The table 164 may include any accessories mounted to or otherwise coupled to the table 164 such as, for example, a bed rail, a bed rail adaptor, an arm rest, an extender, or the like. The table 164 may be stationary or may be operable to maneuver the patient 162 (e.g., the table 164 may be able to move). In some embodiments, the table 164 has two positioning degrees of freedom and one rotational degree of freedom, which allows positioning of the specific anatomy of the patient anywhere in space (within a volume defined by the limits of movement of the table 164). For example, the table 164 can slide forward and backward and from side to side, and can tilt (e.g., around an axis positioned between the head and foot of the table 164 and extending from one side of the table 164 to the other) and/or roll (e.g., around an axis positioned between the two sides of the table 164 and extending from the head of the table 164 to the foot thereof). In other embodiments, the table 164 can bend at one or more areas (which bending may be possible due to, for example, the use of a flexible surface for the table 164, or by physically separating one portion of the table 164 from another portion of the table 164 and moving the two portions independently). In at least some embodiments, the table 164 may be manually moved or manipulated by, for example, a surgeon or other user, or the table 164 may comprise one or more motors, actuators, and/or other mechanisms configured to enable movement and/or manipulation of the table 164 by a processor such as the processor 104.

The imaging apparatus 112 comprises a gantry. The gantry may be or comprise a substantially circular, or “O-shaped,” housing that enables imaging of objects placed into an isocenter of the housing. In other words, the gantry may be positioned around the object being imaged. In some embodiments, the gantry may be disposed at least partially within the upper wall 152, the sidewalls 156A, 156B, and the lower wall 160 of the imaging apparatus 112.

The imaging apparatus 112 also comprises a source 136 and a detector 140. The source 136 may be a device configured to generate and emit radiation, and the detector 140 may be a device configured to detect the emitted radiation. In some embodiments, the source 136 and the detector 140 may be or comprise an imaging source and an imaging detector (e.g., the source 136 and the detector 140 are used to generate data useful for producing images). The source 136 may be positioned in a first position and the detector 140 may be positioned in a second position opposite the source 136. In some embodiments, the source 136 comprises an X-ray source such as, for example, a thermionic emission tube, a cold emission x-ray tube, and/or the like. In other embodiments, the source 136 may comprise a tube that generates and emits radiation without the use of x-rays, such as a nuclear isotope tube that uses nuclear decay of one or more nuclear isotopes to generate radiation. The source 136 may project a radiation beam 176 that passes through the patient 162 and onto the detector 140 located on the opposite side of the imaging apparatus 112. The detector 140 may be or comprise one or more sensors that receive the radiation beam 176 (e.g., once the radiation beam 176 has passed through the patient 162) and transmit information related to the radiation beam 176 to one or more other components of the system 100 for processing, such as to the processor 104. The source 136 and/or the detector 140 may comprise a collimator 144. The collimator 144 may be configured to confine or shape the radiation beam 176 as it is emitted from the source 136 and/or as it is received by the detector 140. Additionally or alternatively, the collimator 144 may filter the radiation beam 176. In other words, the collimator 144 may perform a beam filtration to reduce the x-ray flux (e.g., the amount of x-ray output from the source 136 over a given amount of time). In some cases, the amount of beam filtration may be adjustable, such that the source 136 outputs a constant radiation beam 176 whose x-ray flux through the collimator 144 can be changed based on, for example, a desired amount of modulation when capturing surgical images, as discussed in further detail below.

In some embodiments, the detector 140 may comprise an array. For example, the detector 140 may comprise three 2D flat panel solid-state detectors arranged side-by-side, and angled to approximate the curvature of the imaging apparatus 112. It will be understood, however, that various detectors and detector arrays can be used with the imaging apparatus 112, including any detector configurations used in typical diagnostic fan-beam or cone-beam CT scanners. For example, the detector 140 may comprise a 2D thin-film transistor X-ray detector using scintillator amorphous-silicon technology.

The source 136 may be or comprise a radiation tube (e.g., an x-ray tube) capable of generating the radiation beam 176. The radiation tube may have one or more parameters that are adjustable to control the characteristics of the radiation beam 176. For example, one parameter is the kilovoltage peak (kVp) that may be adjusted to change the acceleration of electrons from the cathode to the anode of the radiation tube, resulting in an increase in the quantity and intensity of the radiation beam 176. Another adjustable parameter includes the tube current (e.g., in mAs) that may be adjusted to change the amount of x-rays or other radiation generated by the radiation tube. Another adjustable parameter includes the pulse width of the radiation beam 176, which reflects how long the source 136 generates the radiation beam 176. In some embodiments, each parameter of the radiation tube may be capable of being independently controlled and adjusted. Such changes in the one or more parameters may result in a different amount of radiation being generated by the radiation tube and emitted by the source 136. By tuning the parameters to account for the attenuation of the radiation beam 176, the imaging apparatus 112 can effectively modulate the radiation beam 176 to generate higher quality reconstructed images and/or minimize patient exposure to radiation, as discussed in further detail below.

The source 136 and the detector 140 may be attached to the gantry and configured to rotate 360 degrees around the patient 162 in a continuous or step-wise manner so that the radiation beam 176 can be projected through the patient 162 at various angles. In other words, the source 136 and the detector 140 may rotate, spin, or otherwise revolve about an axis that passes through the top and bottom of the patient, with the volume of interest positioned at the isocenter of the imaging apparatus 112. The imaging apparatus 112 comprises a drive mechanism 148, the drive mechanism 148 capable of causing the gantry to move such that the source 136 and the detector 140 encircle the patient 162 on the table 164. Additionally or alternatively, the source 136 and the detector 140 may move along a length of the patient 162, as depicted in FIG. 1F. For example, the table 164 holding the patient 162 may move in the direction of arrow 184 while the source 136 and detector 140 remain in a fixed location, such that the length of the patient can be scanned. At each projection angle in the revolution, the radiation beam 176 passes through and is attenuated by the patient 162. The attenuated radiation is then detected by the detector 140. The detected radiation from each of the projection angles can then be processed, using various reconstruction techniques, to produce a 2D or 3D reconstruction image of the patient 162. For example, the processor 104 may be used to perform image processing 120 to generate the reconstruction image.

In some examples, such as those depicted in FIG. 1E, the source 136 and the detector 140 may be aligned asymmetrically from one another. For example, the detector 140 may be shifted off-axis from the source 136 before the imaging apparatus 112 is used to image the patient 162. As a result, the radiation beam 176 may pass through the patient 162 without passing through the isocenter of the imaging apparatus 112, enabling imaging of different portions of the patient 162 that are not located at the isocenter of the imaging apparatus 112. The source 136 and the detector 140 may be configured asymmetrically to account for larger patients, to enable imaging of patient anatomy not located at the isocenter of the patient 162, and/or for any other reason.

In some cases, such as those depicted in FIG. 1C, the source 136 and/or the detector 140 may be positioned in a lateral orientation 172 relative to the patient 162. While in the lateral orientation 172, the source 136 and the detector 140 may be respectively positioned on the right- and left-hand sides of the patient 162 (or vice versa). In some embodiments, the source 136 and the detector 140 may enter the lateral orientation 172 during the course of capturing a 3D scan of the patient 162. In other embodiments, the navigation system 118 may cause the source 136 and the detector 140 to move into the lateral orientation 172 (e.g., using the drive mechanism 148 to move the gantry) to capture an image depicting the lateral view of the patient 162. While in the lateral orientation 172, the radiation beam 176 may experience a relatively higher attenuation as compared to other projection angles due to, for example, the additional anatomical tissue through which the radiation beam 176 must pass before arriving at the detector 140.

In other cases, such as those depicted in FIG. 1D, the source 136 and/or the detector 140 may be positioned in an anterior-posterior (AP) orientation 180. While in the AP orientation 180, the source 136 and the detector 140 may be respectively positioned above and below the patient 162 (or vice versa). In some embodiments, the source 136 and the detector 140 may enter the AP orientation 180 during the course of capturing the 3D scan of the patient 162. In other embodiments, the navigation system 118 may cause the source 136 and the detector 140 to move into the AP orientation 180 (e.g., using the drive mechanism 148 to move the gantry) to capture an image depicting the anterior-posterior view of the patient 162. In contrast to the lateral orientation 172, while in the AP orientation 180 the radiation beam 176 may experience a relatively lower attenuation as compared to other projections angles due to, for example, the smaller amount of anatomical tissue through which the radiation beam 176 must pass before arriving at the detector 140.

The varying amount of attenuation of the radiation beam 176 may affect the quality of the resulting reconstructed image. In cases where the detector 140 receives an insufficient signal, such as when the radiation beam 176 passes through a relatively large amount of anatomical tissue to reach the detector 140, the reconstructed image may have a poor signal to noise ratio and/or may have poor contrast detection. Additionally or alternatively, when the detector 140 receives too strong a signal, such as when the radiation beam 176 passes through a relatively small amount of anatomical tissue to reach the detector 140, the reconstruction image may have degraded contrast due to pixel saturation on the detector 140.

To account for the differences in the radiation beam 176 when the source 136 is in the lateral orientation 172 and the AP orientation 180, the parameters of the source 136 or components thereof (e.g., a radiation tube, the beam filtration of the collimator 144, etc.) may be adjusted depending on the angle of the source 136 and/or the detector 140 relative to the patient 162. For example, when in the lateral orientation 172, the kVp, the pulse width, and/or the mAs of the source 136 may be increased, such that the radiation beam 176 increases in intensity, duration, and/or the like. As another example, when in the AP orientation 180, the kVp, the pulse width, and/or the mAs of the source 136 may be decreased, such that the radiation beam 176 decreases in intensity, duration, and/or the like. The adjustments may effectively modulate the radiation beam 176 to account for varying amounts of attenuation, beneficially improving the quality of the reconstructed image, as discussed in further detail below. It is to be understood that the above discussion of modulation of the parameters of the source 136 may be applied to any multi-dimensional image captured using the source 136 and the detector 140. For example, the parameters of the source 136 may be modulated when the source 136 and the detector 140 revolve around the patient 162 to capture data that can be used to generate one or more 3D images. Additionally or alternatively, the parameters of the source 136 may be modulated when the source 136 and the detector 140 translate laterally across a patient along a patient axis (e.g., an axis parallel to the arrow 184) to capture data that can be used to generate one or more 2D images.

The system 100 or similar systems may be used, for example, to carry out one or more aspects of any of the methods 200, 300, 400, and/or 500 described herein. The system 100 or similar systems may also be used for other purposes.

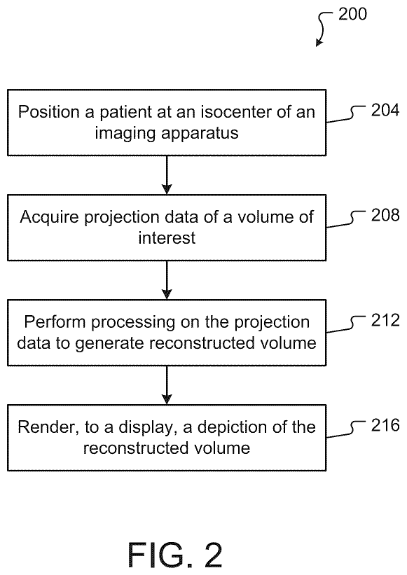

FIG. 2 depicts a method 200 that may be used, for example, to produce one or more 3D images of patient anatomy. The images may be used for treatment planning, surgical navigation, in conjunction with robotics, intraoperative reconciliation, and/or the like.

The method 200 (and/or one or more steps thereof) may be carried out or otherwise performed, for example, by at least one processor. The at least one processor may be the same as or similar to the processor(s) 104 of the computing device 102 described above. The at least one processor may be part of a navigation system (such as a navigation system 118). A processor other than any processor described herein may also be used to execute the method 200. The at least one processor may perform the method 200 by executing elements stored in a memory such as the memory 106. The elements stored in the memory and executed by the processor may cause the processor to execute one or more steps of a function as shown in method 200. One or more portions of a method 200 may be performed by the processor executing any of the contents of memory, such as an image processing 120, a segmentation 122, a transformation 124, and/or a registration 128.

The method 200 comprises positioning a patient at an isocenter of an imaging apparatus (step 204). The patient 162 may be positioned on the table 164 (e.g., in a supine position, in a prone position, etc.), with the table 164 positioned at the isocenter of the imaging apparatus 112. In some embodiments, table 164 may mechanically couple with the imaging apparatus 112 (e.g., the imaging apparatus 112 comprises slots, brackets, mounts, or other components that enable the table 164 to connect to the imaging apparatus 112). The patient 162 and/or the table 164 may then be localized to the imaging apparatus 112 using a variety of different well-known techniques.

The method 200 also comprises acquiring projection data of a volume of interest (step 208). The projection data may be acquired based on signals generated by the detector 140 based on the radiation beam 176 that has passed through the patient 162 before arriving at the detector 140. In some embodiments, the radiation beam 176 may pass through the volume of interest that comprises one or more anatomical elements or other patient anatomy that is the subject of the surgery or surgical procedure. For example, the volume of interest may comprise vertebrae that are the subject of a spinal fusion procedure. In some embodiments, the source 136 and the detector 140 may revolve around an axis that passes through the volume of interest in up to 360 degrees, such that the detector 140 receives the radiation beam 176 at a plurality of different projection angles and generates signals that can be used by the processor 104 to generate a 3D reconstruction of the volume of interest. Additionally or alternatively, the source 136 and the detector 140 may move along a length of the patient (e.g., along the direction of the axis that passes through the volume of interest) and the detector 140 may receive the radiation beam 176. The detector 140 may generate one or more signals based on the received radiation beam 176, which signals may be used by the processor 104 to generate the reconstructed image.

The method 200 also comprises performing processing on the projection data to generate reconstructed volume (step 212). Once the projection data has been received at the detector 140, output signals from the detector 140 may be processed by one or more components of the system 100 (e.g., the computing device 102, the processor 104, etc.) to generate the reconstructed volume. For example, the processor 104 may access image processing 120 that uses one or more iterative reconstruction methods, such as Algebraic Reconstruction Technique (ART), Maximum Likelihood Expectation Maximization (MLEM), Ordered Subset Expectation Maximization (OSEM), or the like to generate the reconstructed volume.

The method 200 also comprises rendering, to a display, a depiction of the reconstructed volume (step 216). Once the reconstruction volume has been generated by the processor 104 or other component of the system 100, a depiction of the reconstruction volume can be rendered to a display (e.g., user interface 110). The reconstruction volume may be rendered preoperatively, intraoperatively, and/or postoperatively. In some embodiments, the user (e.g., a surgeon, a member of surgical staff, etc.) may be able to interact with the depicted reconstruction volume (e.g., rotate the reconstruction volume depiction to view the reconstructed volume at various angles, zoom in/out, etc.). In some embodiments, the reconstruction volume may depict the one or more anatomical elements or other patient anatomy located within the volume of interest of the patient 162.

The present disclosure encompasses embodiments of the method 200 that comprise more or fewer steps than those described above, and/or one or more steps that are different than the steps described above.

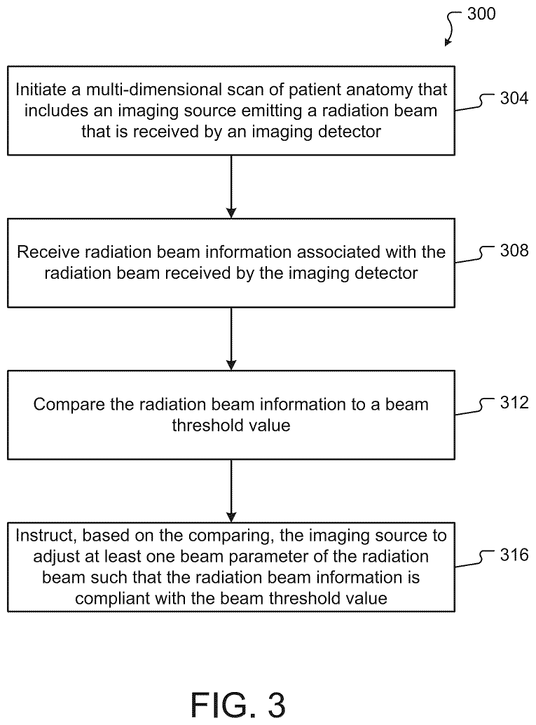

FIG. 3 depicts a method 300 that may be used, for example, to modulate a radiation beam based on attenuation of patient anatomy. In some embodiments, the method 300 may be performed during the course of step 216 of the method 200, where projection data is acquired.

The method 300 (and/or one or more steps thereof) may be carried out or otherwise performed, for example, by at least one processor. The at least one processor may be the same as or similar to the processor(s) 104 of the computing device 102 described above. The at least one processor may be part of a navigation system (such as a navigation system 118). A processor other than any processor described herein may also be used to execute the method 300. The at least one processor may perform the method 300 by executing elements stored in a memory such as the memory 106. The elements stored in memory and executed by the processor may cause the processor to execute one or more steps of a function as shown in method 300. One or more portions of a method 300 may be performed by the processor executing any of the contents of memory, such as an image processing 120, a segmentation 122, a transformation 124, and/or a registration 128.

The method 300 comprises initiating a multi-dimensional scan of patient anatomy that includes an imaging source emitting a radiation beam that is received by an imaging detector (step 304). The multi-dimensional scan may be or comprise a 2D scan and/or a 3D scan of the patient. The 3D scan of the patient anatomy may include the source 136 and the detector 140 revolving around the patient 162 (e.g., using the gantry), while the 2D scan may comprise the source 136 and the detector 140 moving laterally along a length of the patient 162 (e.g., along the direction of the arrow 184). In some embodiments, the source 136 and the detector 140 may move in a continuous or step-wise manner relative to the patient 162.

The method 300 also comprises receiving radiation beam information associated with the radiation beam received by the imaging detector (step 308). As the source 136 and the detector 140 move relative to the patient 162, the processor 104 may receive projection data in the form of a signal output from the detector 140. The radiation beam information may be or comprise the signals output from the detector 140 at any particular angle of the detector 140 relative to the patient 162 during the 3D scan. In some embodiments, the radiation beam information may be continuously output from the detector 140 as the detector 140 revolves around the patient 162.

The method 300 also comprises comparing radiation beam information to a beam threshold value (step 312). The beam threshold value may represent a value of the desired signal level of the radiation beam 176 received at the detector 140. The beam threshold may be based on the type of surgery or surgical procedure, the type of source 136 and/or detector 140 used, user preference, surgical plan information and/or clinical objective (e.g., the beam threshold may be set to a low value to minimize the radiation dose to the patient 162), combinations thereof, and/or the like. In some embodiments, the beam threshold may be predetermined and/or set based on information retrieved from the database 130. In some embodiments, the beam threshold value may be based at least partially on a configuration of the detector 140. In other words, the detector 140 may be configured for an imaging mode, with the detector 140 capable of receiving the radiation beam 176 and outputting a signal sufficient for use in performing an image reconstruction. The signal may be deemed sufficient when the desired signal level falls between maximum and minimum signal values that are received at the detector 140, which signal values may be known or predetermined. In such embodiments, the beam threshold may comprise the maximum value of the signal amplitude received at the detector 140. Additionally or alternatively, the beam threshold may be or comprise the minimum value of the signal amplitude received at the detector 140. In some embodiments, the beam threshold may comprise a plurality or range of values (e.g., the beam threshold comprises both the maximum and minimum value of the signal amplitude received at the detector 140).

The radiation beam information for a particular projection may be compared to the beam threshold value. In some embodiments, the beam threshold value may comprise the range and/or boundaries of the signal level capable of being received at the detector 140 (such as when the detector 140 receives the radiation beam 176). When the radiation beam information (e.g., the signal received at the detector 140 for a particular projection) exceeds the beam threshold value, the radiation beam 176 may be less attenuated than desired, such as when the source 136 and the detector 140 are in the AP orientation 180 and the radiation beam 176 passes through a relatively smaller amount of matter. As a result, the pixels of the detector 140 may be saturated and may be outputting higher readings than desired. Conversely, when the radiation beam information falls below the beam threshold value, the radiation beam 176 may more attenuated than desired, such as when the source 136 and the detector 140 are in the lateral orientation 172 and the radiation beam 176 passes through a relatively greater amount of matter. As a result, the detector 140 readings may contain a greater amount of noise than desired. In some embodiments, the radiation beam information may be compared to the beam threshold value for each projection as the source 136 and the detector 140 scan the patient 162.

The method 300 also comprises instructing, based on the comparing, the imaging source to adjust at least one beam parameter of the radiation beam such that the radiation beam information is compliant with the beam threshold value (step 316). The radiation beam information may be compliant with the beam threshold value when the output signal from the detector 140 falls between the maximum and minimum boundaries on the signal level represented by the beam threshold value. For example, when the output signal from the detector 140 exceeds the maximum signal level boundary, the radiation beam information may be treated as having exceeded the beam threshold value. Similarly, when the output signal from the detector 140 falls below the minimum signal level boundary, the radiation beam information may be treated as having fallen below the beam threshold value.

One or more beam parameters of the radiation beam 176 may be adjusted to affect the output readings from the detector 140 such that the signal readings from the detector 140 fall within the minimum and maximum boundary values represented by the beam threshold value. For example, when the signal reading falls below the minimum boundary value, the kVp, the pulse width, and/or the mAs of the emission tube may be increased, such that the intensity and/or duration of the radiation beam 176 increases at the particular projection. Additionally or alternatively, the pulse width of the emission tube may be changed to increase the intensity and/or duration of the radiation beam 176. As another example, when the signal reading exceeds the maximum boundary value, the kVp, the pulse width, and/or the mAs of the emission tube may be decreased, such that the intensity and/or duration of the radiation beam 176 decreases at the particular projection. In yet another example, when the signal reading exceeds the maximum boundary value, the collimator 144 may be adjusted such that the flux of the radiation beam 176 decreases.

In some embodiments, the adjustment of the beam parameters may be calculated by the system 100 (e.g., using the processor 104), but not initially implemented at the particular projection. This may occur, for example, when the 3D scan progresses faster than the adjustment of the radiation beam 176 can occur (e.g., the gantry moves the source 136 and the detector 140 faster than the radiation beam 176 can be adjusted). However, since the second 180 degree revolution of the patient 162 has the same projection angles as the first 180 degree revolution (e.g., due to the symmetry of the circular scan), the adjustment of the beam parameters may be applied to the projection during the second 180 degree revolution.

The present disclosure encompasses embodiments of the method 300 that comprise more or fewer steps than those described above, and/or one or more steps that are different than the steps described above.

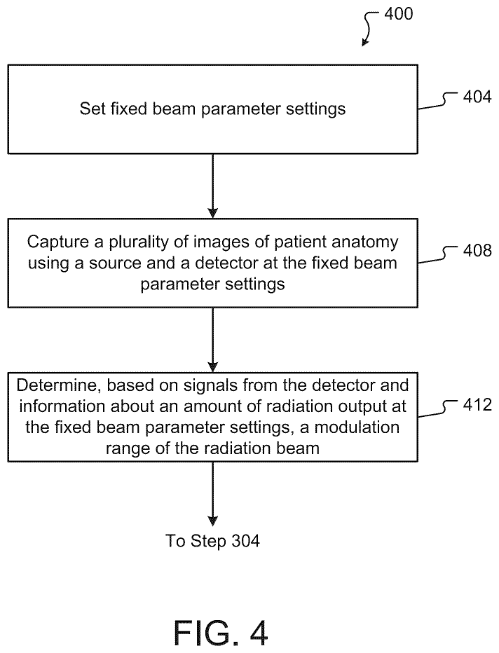

FIG. 4 depicts a method 400 that may be used, for example, to determine a modulation range of a radiation beam 176 based on anterior-posterior view images and lateral view images of patient anatomy.

The method 400 (and/or one or more steps thereof) may be carried out or otherwise performed, for example, by at least one processor. The at least one processor may be the same as or similar to the processor(s) 104 of the computing device 102 described above. The at least one processor may be part of a navigation system (such as a navigation system 118). A processor other than any processor described herein may also be used to execute the method 400. The at least one processor may perform the method 400 by executing elements stored in a memory such as the memory 106. The elements stored in memory and executed by the processor may cause the processor to execute one or more steps of a function as shown in method 400. One or more portions of a method 400 may be performed by the processor executing any of the contents of memory, such as an image processing 120, a segmentation 122, a transformation 124, and/or a registration 128.

The method 400 comprises setting fixed beam parameter settings (step 404). The fixed beam parameter settings may be based on the default settings of the source 136. In some embodiments, the fixed beam parameter settings may comprise a fixed kVp, pulse width, and/or mAs setting on the radiation tube of the source 136. In some embodiments, the fixed beam parameter settings may be stored in the database 130. The processor 104 may control the radiation tube to emit the radiation beam 176 at the fixed beam parameter settings.

The method 400 also comprises capturing a plurality of images of patient anatomy using a source and a detector at the fixed beam parameter settings (step 408). The plurality of images may comprise an anterior-poster view image and/or a lateral view image of the patient 162. In some embodiments, the capturing may occur before the imaging apparatus 112 performs the 3D image acquisition of the patient 162. Based on the signal output of the detector 140 as well as information about an amount of radiation output by the source 136, the processor 104 may determine the relative difference in attenuation and modulation ranges for the radiation tube. For example, the signal output of the detector 140 at the anterior-posterior view may indicate the minimum amount of attenuation experienced by the radiation beam 176, while the signal output of the detector 140 at the lateral view may indicate the maximum amount of attenuation experienced by the radiation beam 176.

The method 400 also comprises determining, based on signals from the detector at the fixed beam parameter settings, a modulation range of the radiation beam (step 412). Based on the minimum and maximum attenuation values determined in the step 408 and an amount of radiation output by the source 136 (e.g., determined based on the operating parameters of the radiation tube used by the source 136), the processor 104 may determine a relative modulation range of the radiation tube. For example, the processor 104 may determine a minimum intensity, dose, duration, or the like of the radiation beam 176 based on the minimum amount of attenuation, and may determine a maximum intensity, dose, duration, or the like of the radiation beam 176 based on the maximum amount of attenuation. The modulation range may thus fall between the minimum and maximum values.

In some embodiments, the processor 104 may use the modulation range when adjusting the radiation beam in the step 316. For example, the processor 104 may determine that the output signal from the detector 140 exceeds the maximum beam threshold value, and may use the modulation range to determine an appropriate modulation to the kVp of the radiation beam 176 such that the output signal from the detector 140 falls below the maximum beam threshold value. Conversely, the processor 104 determine that the output signal from the detector 140 falls below the minimum beam threshold value, and may use the modulation range to determine an appropriate adjustment to the kVp of the radiation beam 176 such that the output signal of the detector 140 exceeds the minimum beam threshold value. In some cases, the parameters of the radiation beam 176 may remain the same, but one or more parameters of the collimator 144 may be adjusted to change the output signal on the detector 140. For example, a beam filtration parameter-such as a pose of the collimator relative to the source 136 or components thereof and/or relative to the detector 140—may be adjusted such that the flux of the radiation beam 176 changes, resulting in a corresponding change in the output signal from the detector 140.

In some embodiments, the beam threshold values may comprise a plurality of beam threshold values that are set per projection for both 2D and 3D modulation. In some cases, the detector 140 may have a plurality of regions of interest (ROI) defined within the field of view of the detector 140, and a minimum beam threshold value and a maximum beam threshold value may be set for each ROI. The detector 140 may have a two, three, four, five, or more ROIs. For example, a 2D scan of the patient 162 may have three ROIs. As a result, the beam threshold values may comprise a maximum beam threshold value and a minimum beam threshold value for each of the three ROIs, for a total of six beam threshold values. Given the larger number of beam threshold values, the beam threshold values may be set and the modulation may be adjusted based on acquisition type, anatomy type, beam collimation requirements, combinations thereof, and/or the like. Continuing the 2D long film example, the processor 104 may adjust the radiation beam such that the output signal from the detector 140 is compliant with all six beam threshold values. Alternatively, the processor 104 may prioritize ensuring the radiation beam is compliant with the beam threshold values for one of the ROIs, such as when the ROI overlaps with one or both of the other ROIs.

In some embodiments, the modulation range of the radiation beam 176 may be configured such that the image quality at any given projection angle is optimized, while the patient exposure to radiation is minimized. In some embodiments, the step 412 may proceed to the step 304 of the method 300.

The present disclosure encompasses embodiments of the method 400 that comprise more or fewer steps than those described above, and/or one or more steps that are different than the steps described above.

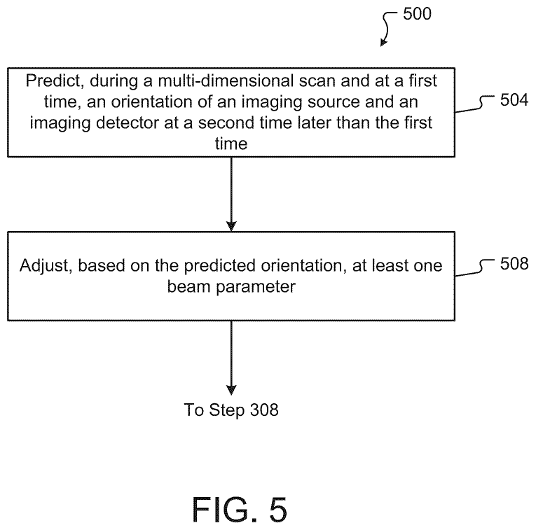

FIG. 5 depicts a method 500 that may be used, for example, to perform real-time adjustment of beam parameters based on a predicted orientation at a later time.

The method 500 (and/or one or more steps thereof) may be carried out or otherwise performed, for example, by at least one processor. The at least one processor may be the same as or similar to the processor(s) 104 of the computing device 102 described above. The at least one processor may be part of a navigation system (such as a navigation system 118). A processor other than any processor described herein may also be used to execute the method 500. The at least one processor may perform the method 500 by executing elements stored in a memory such as the memory 106. The elements stored in memory and executed by the processor may cause the processor to execute one or more steps of a function as shown in method 500. One or more portions of a method 500 may be performed by the processor executing any of the contents of memory, such as an image processing 120, a segmentation 122, a transformation 124, and/or a registration 128.

The method 500 comprises predicting, during a multi-dimensional scan and at a first time, an orientation of an imaging source and an imaging detector at a second time later than the first time (step 504). The multi-dimensional scan may comprise a 3D scan where the source 136 and the detector 140 revolve around the patient 162, or may comprise a 2D scan where the source 136 and the detector 140 translate along a length of the patient 162. As the source 136 and the detector 140 move relative to the patient 162, the processor 104 may check each projection to ensure compliance with the beam threshold value. Additionally or alternatively, the current projection may be used to predict and adjust in real-time the parameters of the radiation beam 176 such that the radiation beam 176 is compliant with the beam threshold value at the next projection. In some embodiments, the source 136 and the detector 140 may be positioned at a first angle relative to the patient 162. Based on the readout from the detector 140, the processor 104 may use image processing 120 to generate a reconstructed 2D image of the patient 162. In some embodiments, the readout from the detector 140 corresponds to a region of interest may be used in generating the reconstructed image. The region of interest may change depending on the patient anatomy being imaged. For example, when imaging the chest of the patient, the region of interest may include signal output from the center of the detector 140 as well as from peripheral portions of the detector 140. In another example, when imaging the head of the patient, signal output from the center of the detector 140 may be used without readings from the peripheral portions. In this example, the processor 104 may use the signal readouts from the center of the detector 140 in reconstructing the 2D image.

The processor 104 may then use segmentation 122 to segment the reconstructed image and to identify patient anatomy. Based on the orientation of the patient anatomy depicted in the segmented image, the processor 104 may use transformation 124 and/or registration 128 to determine the pose of the anatomy depicted in the segmented image relative to the source 136 and the detector 140. The processor 104 may then predict the next orientation of the source 136 and the detector 140 relative to the patient based on the current pose of the source 136 and the detector 140.

The method 500 also comprises adjusting, based on the predicted orientation, at least one beam parameter (step 508). The beam parameters may be adjusted differently based on the predicted orientation. For example, when the processor 104 predicts that the source 136 and the detector 140 are moving toward a projection that will have greater attenuation, such as when the source 136 and the detector 140 are moving into the lateral orientation 172 at the second time, the beam parameters may be adjusted such that the radiation beam 176 increases in intensity, duration, and/or the like. As another example, when the processor 104 predicts that the source 136 and the detector 140 are moving toward a projection that will have less attenuation, such as when the source 136 and the detector 140 are moving into the AP orientation 180 at the second time, the beam parameters may be adjusted such that the radiation beam 176 decreases in intensity, duration, and/or the like. In some embodiments, the step 508 may proceed to the step 308 of the method 300 where, for example, the radiation beam information for the projection captured at the second time may be received.

The present disclosure encompasses embodiments of the method 500 that comprise more or fewer steps than those described above, and/or one or more steps that are different than the steps described above.

As noted above, the present disclosure encompasses methods with fewer than all of the steps identified in FIGS. 2, 3, 4, and 5 (and the corresponding description of the methods 200, 300, 400, and 500), as well as methods that include additional steps beyond those identified in FIGS. 2, 3, 4, and 5 (and the corresponding description of the methods 200, 300, 400, and 500). The present disclosure also encompasses methods that comprise one or more steps from one method described herein, and one or more steps from another method described herein. Any correlation described herein may be or comprise a registration or any other correlation.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

The techniques of this disclosure may also be described in the following examples.

Example 1: A system (100), comprising:
an imaging source (136);
an imaging detector (140);
a processor (104); and
a memory (106) coupled to the processor (104) and storing data thereon that, when executed by the processor (104), enable the processor (104) to:
initiate a multi-dimensional scan of patient anatomy that includes the imaging source (136) emitting a radiation beam (176) that is received by the imaging detector (140);
receive radiation beam information associated with the radiation beam (176) received by the imaging detector (140);
compare the radiation beam information to a beam threshold value; and
instruct, based on the comparing, the imaging source (136) to adjust at least one beam parameter of the radiation beam (176) such that the radiation beam information is compliant with the beam threshold value.

Example 2: The system according to example 1, wherein the data, when processed by the processor (104), further enable the processor (104) to:
capture a plurality of images of the patient anatomy using the imaging source (136) and the imaging detector (140) at fixed beam parameter settings; and
determine, based on signals from the imaging detector (140) and information about an amount of radiation output at the fixed beam parameter settings, a modulation range of the radiation beam (176).

Example 3: The system according to example 2, wherein the plurality of images comprises an anterior-posterior view image and a lateral view image of the patient anatomy.

Example 4: The system according to any of examples 1 to 3, wherein the data, when processed by the processor (104), further enable the processor (104) to:
predict, during the multi-dimensional scan and at a first time, an orientation of the imaging source (136) and the imaging detector (140) at a second time later than the first time; and
adjust, based on the predicted orientation, the at least one beam parameter.

Example 5: The system according to any of examples 1 to 4, wherein the imaging source (136) comprises a thermionic emission tube, a cold emission tube, or a nuclear isotope tube.

Example 6: The system according to any of examples 1 to 5, wherein the at least one beam parameter comprises at least one of an amplitude and an amount of current.

Example 7: The system according to any of examples 1 to 6, wherein the multi-dimensional scan comprises at least one of the imaging source (136) and the imaging detector (140) revolving about a first axis that passes through the patient anatomy and the imaging source (136) and the imaging detector (140) moving along a direction of the first axis.

Example 8: The system according to any of examples 1 to 7, wherein the beam threshold value is based on a configuration of the imaging detector (140).

Example 9: The system according to any of examples 1 to 8, wherein the at least one beam parameter is adjusted by changing a beam filtration parameter of a collimator (144) connected to the imaging source (136).

Example 10: A system, comprising:
a processor (104); and
a memory (106) coupled to the processor (104) and storing data thereon that, when processed by the processor (104), enable the processor (104) to:
initiate a multi-dimensional scan of patient anatomy that includes a source (136) emitting a radiation beam (176) that is received by a detector (140);
receive radiation beam information associated with the radiation beam (176);
compare the radiation beam information to a beam threshold; and
instruct, based on the comparing, the source (136) to adjust at least one beam parameter of the radiation beam (176) such that the radiation beam information is compliant with the beam threshold.

Example 11: The system according to example 10, wherein the data, when processed by the processor (104), further enable the processor (104) to:
capture a plurality of images of the patient anatomy using the source (136) and the detector (140) at fixed beam parameter settings; and
adjust, based on signals from the detector (140) and information about an amount of radiation output at the fixed beam parameter settings, a modulation range of the radiation beam (176).

Example 12: The system according to example 11, wherein the plurality of images comprises an anterior-posterior view image and a lateral view image of the patient anatomy.

Example 13: The system according to any of examples 10 to 12, wherein the data, when processed by the processor (104), further enable the processor (104) to:
predict, during the multi-dimensional scan and at a first time, an orientation of the source (136) and the detector (140) at a second time later than the first time; and
adjust, based on the predicted orientation, the at least one beam parameter.

Example 14: The system according to any of examples 10 to 13, wherein the beam threshold is based on a configuration of the detector (140).

Example 15: The system according to any of examples 10 to 14, wherein the at least one beam parameter is adjusted by changing a beam filtration parameter on a collimator (144) connected to the source (136).

Various examples of the disclosure have been described. These and other examples are within the scope of the following claims.

What is claimed is:

1. A system, comprising:
an imaging source;
an imaging detector;
a processor; and
a memory coupled to the processor and storing data thereon that, when executed by the processor, enable the processor to:
initiate a multi-dimensional scan of patient anatomy that includes the imaging source emitting a radiation beam that is received by the imaging detector;
receive radiation beam information associated with the radiation beam received by the imaging detector;
compare the radiation beam information to a beam threshold value; and
instruct, based on the comparing, the imaging source to adjust at least one beam parameter of the radiation beam such that the radiation beam information is compliant with the beam threshold value.

2. The system of claim 1, wherein the data, when processed by the processor, further enable the processor to:
capture a plurality of images of the patient anatomy using the imaging source and the imaging detector at fixed beam parameter settings; and
determine, based on signals from the imaging detector and information about an amount of radiation output at the fixed beam parameter settings, a modulation range of the radiation beam.

3. The system of claim 2, wherein the plurality of images comprises an anterior-posterior view image and a lateral view image of the patient anatomy.

4. The system of claim 1, wherein the data, when processed by the processor, further enable the processor to:
predict, during the multi-dimensional scan and at a first time, an orientation of the imaging source and the imaging detector at a second time later than the first time; and
adjust, based on the predicted orientation, the at least one beam parameter.

5. The system of claim 1, wherein the imaging source comprises a thermionic emission tube, a cold emission tube, or a nuclear isotope tube.

6. The system of claim 1, wherein the at least one beam parameter comprises at least one of an amplitude and an amount of current.

7. The system of claim 1, wherein the multi-dimensional scan comprises at least one of the imaging source and the imaging detector revolving about a first axis that passes through the patient anatomy and the imaging source and the imaging detector moving along a direction of the first axis.

8. The system of claim 1, wherein the beam threshold value is based on a configuration of the imaging detector.

9. A system, comprising:
a processor; and
a memory coupled to the processor and storing data thereon that, when processed by the processor, enable the processor to:
initiate a multi-dimensional scan of patient anatomy that includes a source emitting a radiation beam that is received by a detector;
receive radiation beam information associated with the radiation beam;
compare the radiation beam information to a beam threshold; and
instruct, based on the comparing, the source to adjust at least one beam parameter of the radiation beam such that the radiation beam information is compliant with the beam threshold.

10. The system of claim 9, wherein the data, when processed by the processor, further enable the processor to:
capture a plurality of images of the patient anatomy using the source and the detector at fixed beam parameter settings; and
adjust, based on signals from the detector and information about an amount of radiation output at the fixed beam parameter settings, a modulation range of the radiation beam.

11. The system of claim 10, wherein the plurality of images comprises an anterior-posterior view image and a lateral view image of the patient anatomy.

12. The system of claim 9, wherein the data, when processed by the processor, further enable the processor to:
predict, during the multi-dimensional scan and at a first time, an orientation of the source and the detector at a second time later than the first time; and
adjust, based on the predicted orientation, the at least one beam parameter.

13. The system of claim 9, wherein the source comprises a thermionic emission tube, a cold emission tube, or a nuclear isotope tube.

14. The system of claim 9, wherein the at least one beam parameter comprises at least one of an amplitude and an amount of current.

15. The system of claim 9, wherein the multi-dimensional scan comprises at least one of the source and the detector revolving about a first axis that passes through the patient anatomy and the source and the detector moving along a direction of the first axis.

16. The system of claim 9, wherein the beam threshold is based on a configuration of the detector.

17. An apparatus, comprising:
a source that emits a radiation beam;
a detector;
a processor coupled to at least one of the source and the detector; and
a memory coupled to the processor and storing data thereon that, when executed by the processor, enable the processor to:
initiate a multi-dimensional scan of patient anatomy that includes the detector receiving the radiation beam after the radiation beam has passed through the patient anatomy;
receive radiation beam information associated with the radiation beam received at the detector;
compare the radiation beam information to a beam threshold value; and
instruct, based on the comparing, the source to adjust at least one parameter of the radiation beam such that the radiation beam information is compliant with the beam threshold value.

18. The apparatus of claim 17, wherein the data, when processed by the processor, further enable the processor to:
capture a plurality of images of the patient anatomy using the source and the detector at fixed beam parameter settings; and
determine, based on signals from the detector and information about an amount of radiation output at the fixed beam parameter settings, a modulation range of the radiation beam.

19. The apparatus of claim 18, wherein the plurality of images comprises an anterior-posterior view image and a lateral view image of the patient anatomy.

20. The apparatus of claim 17, wherein the data, when processed by the processor, further enable the processor to:
predict, during the multi-dimensional scan and at a first time, an orientation of the source and the detector at a second time later than the first time; and
adjust, based on the predicted orientation, the at least one parameter.

21. The apparatus of claim 17, wherein the at least one parameter is adjusted by changing a beam filtration parameter of a collimator connected to the source.

22. The system of claim 1, wherein the at least one beam parameter is adjusted by changing a beam filtration parameter of a collimator connected to the imaging source.

23. The system of claim 9, wherein the at least one beam parameter is adjusted by changing a beam filtration parameter on a collimator connected to the source.
